(19) United States
(12) Patent Application Publication
(10) Pub. No.: US 2021/0052633 A1
(43) Pub. Date: Feb. 25, 2021

(54) HYALURONIC ACID SYNTHESIS PROMOTER, METHOD FOR PROMOTING HYALURONIC ACID SYNTHESIS, AND CELL EVALUATION METHOD

(71) Applicant: SEIKAGAKU CORPORATION, Tokyo (JP)

(72) Inventors: Tomochika KISUKEDA, Tokyo (JP); Keiji YOSHIOKA, Tokyo (JP)

(73) Assignee: SEIKAGAKU CORPORATION, Tokyo (JP)

(21) Appl. No.: 16/982,847

(22) PCT Filed: Mar. 26, 2019
(86) PCT No.: PCT/JP2019/012681

§ 371 Date: Sep. 21, 2020

(30) Foreign Application Priority Data
Mar. 27, 2018 (JP) 2018-059772

(51) Int. Cl. A61K 31/728; A61K 31/727; A61K 31/737; A61P 19/02

(52) U.S. Cl. CPC A61K 31/728; A61P 19/02; A61K 31/737; A61K 31/727

(57) ABSTRACT

The present application provides: a technique for promoting hyaluronic acid synthesis by hyaluronic acid-producing cells; a method for evaluating the responsiveness of hyaluronic acid-producing cells to a compound; or a method for evaluating the responsiveness of a polysaccharide derivative represented by formula 1 in the description or a salt thereof to hyaluronic acid-producing cells. By bringing a polysaccharide derivative represented by formula 1 in the description or a salt thereof into contact with hyaluronic acid-producing cells, promotion of hyaluronic acid synthesis is achieved in the hyaluronic acid-producing cells. Further, by using the polysaccharide derivative or a salt thereof for hyaluronic acid-producing cells, the responsiveness of the hyaluronic acid-producing cells can be evaluated using the production of hyaluronic acid as an index. Further, by using hyaluronic acid-producing cells, the responsiveness of the polysaccharide derivative or a salt thereof can be evaluated using the production of hyaluronic acid as an index.

### TECHNICAL FIELD

The present invention relates to a technique for promoting hyaluronic acid synthesis by hyaluronic acid-producing cells.

### BACKGROUND ART

Hyaluronic acid is a type of glycosaminoglycan constituted by a basic backbone, which has a structure composed of N-acetyl-D-glucosamine and D-glucuronic acid that are linked via a β-(1,3) linkage as a disaccharide unit (constituent disaccharide unit), and in which the constituent disaccharide units are repeatedly linked via a β-(1,4) linkage. Hyaluronic acid is widely distributed in whole body tissues such as cartilage, synovial fluid (joint fluid) of the joint cavity, umbilical cord, serum, urine, and the vitreous body of the eye in the living body, and is particularly abundant in synovial fluid. At the cellular level, almost all cells in the living body have a function of synthesizing hyaluronic acid, and hyaluronic acid is considered to be an important substance for living organisms. In the joint, hyaluronic acid plays a role in maintaining the lubrication property of the joint fluid in both dynamic and static situations. Hyaluronic acid also plays various physiological roles such as a role in reducing proinflammatory cytokine production to mitigate cartilage degeneration, and a role in suppressing COX-2 production to attenuate pain in the joint.

In Non Patent Literature 1, it is reported that the content of hyaluronic acid is low or the molecular weight of hyaluronic acid is decreased in the joint fluid of patients with osteoarthritis (hereinafter also referred to as “OA” in the description) or patients with rheumatoid arthritis (hereinafter also referred to as “RA” in the description) as compared with hyaluronic acid in the joint fluid of healthy individuals.

### CITATION LIST

### Patent Literature

Patent Literature 1: WO 2005/066214

### Non Patent Literature

Non Patent Literature 1: Dahl L B, Dahl I M, Engstrom-Laurent A, and Granath K. Concentration and molecular weight of sodium hyaluronate in synovial fluid from patients with rheumatoid arthritis and other arthropathies. Ann. Rheum. Dis. 1985; 44 (12): 817-22

### SUMMARY OF INVENTION

An object of the present invention is to provide a technique for promoting hyaluronic acid synthesis by hyaluronic acid-producing cells.

Another object of the present invention is to provide a method for evaluating the responsiveness of hyaluronic acid-producing cells to a compound.

Another object of the present invention is to provide a method for evaluating the responsiveness of a polysaccharide derivative represented by the below-mentioned formula 1 or a salt thereof to hyaluronic acid-producing cells.

The present inventors found that by bringing a polysaccharide derivative represented by the below-mentioned formula 1 or a salt thereof into contact with hyaluronic acid-producing cells, the synthesis of hyaluronic acid can be promoted in the hyaluronic acid-producing cells, and thus completed the present invention.

One aspect of the present invention relates to use of a polysaccharide derivative having a predetermined structure or a salt thereof for promotion of hyaluronic acid synthesis.

Another aspect of the present invention relates to a method for evaluating the responsiveness of hyaluronic acid-producing cells using a polysaccharide derivative represented by the below-mentioned formula 1 or a salt thereof.

### BRIEF DESCRIPTION OF DRAWINGS

FIG. 1A shows results of evaluating the effect of a polysaccharide derivative (0.1% compound 1) of formula 1 on the molecular weight of hyaluronic acid produced by synovial cells.

FIG. 1B shows results of evaluating the effect of the polysaccharide derivative (0.01% compound 1) of the formula 1 on the molecular weight of hyaluronic acid produced by synovial cells.

FIG. 2 shows results indicating that in a process for culturing synovial cells in a culture medium containing the polysaccharide derivative of the formula 1, a high molecular weight substance whose production was promoted in the cells is hyaluronic acid. In the drawing, panels A and B show results when a hyaluronidase treatment was not performed (black circles) and when the treatment was performed (white circles), respectively.

FIG. 3 shows results indicating a relationship between the culture time of synovial cells in a culture medium containing the polysaccharide derivative of the formula 1 and the molecular weight of hyaluronic acid produced in the cells. In the drawing, panels A to C show results when performing no treatment, when performing a hyaluronic acid (HA) treatment, and when performing a compound 1 treatment in this order, respectively.

FIG. 4A shows results of evaluating the effect of various compounds on the molecular weight of hyaluronic acid produced by synovial cells.

FIG. 4B shows results of evaluating the effect of the concentration of diclofenac sodium (DF-Na) in a culture medium on the molecular weight of hyaluronic acid produced by synovial cells.

FIG. 5A shows results of evaluating the effect of the polysaccharide derivative of the formula 1 on the molecular weight of hyaluronic acid produced by synovial cells derived from a patient with rheumatism.

FIG. 5B shows results of evaluating the effect of the polysaccharide derivative of the formula 1 on the molecular weight of hyaluronic acid produced by synovial cells derived from a patient with osteoarthritis.

FIG. 5C shows results of evaluating the effect of the polysaccharide derivative of the formula 1 on the molecular weight of hyaluronic acid produced by synovial cells derived from a patient with osteoarthritis.

FIG. 5D shows results of evaluating the effect of the polysaccharide derivative of the formula 1 on a cell count after cell culture of synovial cells derived from a patient with osteoarthritis.

FIG. 6 shows results of evaluating the effect of the polysaccharide derivative of the formula 1 on the expression level of a gene involved in hyaluronic acid synthesis or hyaluronic acid degradation. Panel A shows the expression levels of genes HAS1 to HAS3 involved in hyaluronic acid synthesis, and panel B shows the expression levels of genes HYAL1 to HYAL3 involved in hyaluronic acid degradation.

FIG. 7 shows results of evaluating the effect of the polysaccharide derivative of the formula 1 on hyaluronic acid synthesis in a rabbit joint.

### DESCRIPTION OF EMBODIMENTS

According to the present invention, a technique for promoting hyaluronic acid synthesis by hyaluronic acid-producing cells is provided.

According to the present invention, further, a method for evaluating the responsiveness of hyaluronic acid-producing cells to a compound is provided.

Hereinafter, embodiments of the present invention will be described, however, the present invention is not limited only to the following embodiments.

In the description, “hyaluronic acid or a salt thereof” is also simply referred to as “HA”.

In the description, “an effective amount” and “as an active ingredient” mean an amount of a component sufficient for obtaining desired response without causing an excessive adverse event in commensurate with a reasonable risk/benefit ratio. A person skilled in the art would also be able to determine the effective amount in other cases based on the results of one or more specific test examples and technical knowledges without requiring individual tests with respect to each combination of various factors.

One aspect of the present invention relates to a hyaluronic acid synthesis promoter containing an effective amount of a polysaccharide derivative represented by the following formula 1 or a salt thereof:

[Chem. 1]

XA)ₙ formula 1

wherein X is a residue derived from a polysaccharide having at least one of a carboxy group and a hydroxy group; A is a substituent; n is an introduction ratio of the substituent A and is not less than 1 mol % and not more than 80 mol %; between X and A is a bond of the carboxy group or the hydroxy group and the substituent A, and the bond is selected from the group consisting of an ester, a thioester, and an amide; and the substituent A is represented by the following formula 2:

[Chem. 2]

*—Y—Z formula 2

wherein Y is a spacer residue or an ester bond; Z is a diclofenac residue; when Y is a spacer residue, the bond between Y and Z is selected from the group consisting of an ester, a thioester, and an amide; and * is a binding site to X.

As the polysaccharide from which a polysaccharide residue in the polysaccharide derivative of the formula 1 according to the present invention is derived, a polysaccharide having at least one of a carboxy group and a hydroxy group is used. In the polysaccharide derivative according to the present invention, at least part of the carboxy groups and/or the hydroxy groups of the polysaccharide and the substituent A form a covalent bond.

The polysaccharide derivative in the description may be in the form of a salt. Examples of the salt include, but not particularly limited to, metal salts such as a sodium salt, a potassium salt, a calcium salt, a magnesium salt, and a barium salt; an ammonium salt; amine salts such as a methylamine salt, a diethylamine salt, an ethylenediamine salt, a cyclohexylamine salt, and an ethanolamine salt; inorganic acid salts such as a hydrochloride salt, a sulfate salt, a hydrogen sulfate salt, a nitrate salt, a phosphate salt, a hydrobromide salt, and a hydroiodide salt; and organic acid salts such as an acetate salt, a phthalate salt, a fumarate salt, a maleate salt, an oxalate salt, a succinate salt, a methanesulfonate salt, a p-toluenesulfonate salt, a tartrate salt, a hydrogen tartrate salt, and a malate salt. The salt of the polysaccharide derivative is preferably an alkali metal salt (for example, a sodium salt or a potassium salt), more preferably a sodium salt.

Examples of the polysaccharide include, but not limited to, hyaluronic acid, chondroitin, chondroitin sulfate, heparin, heparan sulfate, and carboxy C₁₋₄ alkyldextran (for example, carboxymethyl dextran). The polysaccharide is preferably hyaluronic acid.

The polysaccharide can be used even if it is obtained by any method such as a purified product derived from an animal or a microorganism or a synthesized product by chemical synthesis or the like.

The average molecular weight of the polysaccharide derivative and the polysaccharide from which the polysaccharide residue thereof is derived is not particularly limited, but is exemplified by not less than 10,000 and not more than 5,000,000, and is preferably not less than 500,000 and not more than 3,000,000, more preferably not less than 600,000 and not more than 3,000,000, and further more preferably not less than 600,000 and not more than 1,200,000. Note that in the description, the “average molecular weight” of the polysaccharide derivative and the polysaccharide from which the polysaccharide residue thereof is derived refers to a weight average molecular weight measured by an intrinsic viscosity method.

In the formula 1, n is an introduction ratio of the substituent A, that is, a ratio of the number of substituents A to the number of constituent saccharide units, and is not less than 1 mol % and not more than 80 mol %. The introduction ratio of the substituent A is preferably not less than 5 mol % and not more than 50 mol %, more preferably not less than 10 mol % and not more than 30 mol %, and further more preferably not less than 15 mol % and not more than 30 mol %.

Here, the “introduction ratio” in the description is a value calculated by the following calculation formula 1, and can be determined by, for example, measurement of an absorbance. The introduction ratio is obtained by applying the number of moles per saccharide unit calculated by a carbazole absorbance method and the number of moles of diclofenac of the substituent A calculated from a calibration curve prepared beforehand using an absorbance characteristic of diclofenac to the following calculation formula 1. The introduction ratio can be adjusted by changing a condensing agent, a condensing aid, the reaction equivalent of a spacer molecule, the reaction equivalent of the substituent A, or the like in a step of introduction reaction of the substituent A into the polysaccharide. Note that the “constituent saccharide unit” in the calculation formula 1 refers to, for example, in the case of a polysaccharide having a disaccharide unit as a constituent saccharide unit such as hyaluronic acid, the constituent disaccharide unit.

[Math. 1]

Introduction ratio(mol %)=[number of substituents A/number of constituent saccharide units]×100 Calculation formula 1:

In the formula 1, between X and A is a bond of at least one of the carboxy group and the hydroxy group of the polysaccharide and the substituent A, and the bond is selected from the group consisting of an ester, a thioester, and an amide. Preferably, the bond between X and A is an ester or an amide. When a spacer residue is not contained between X and A, and X and Z are directly bonded to each other, the bond between X and A is an ester. When X and A are bonded through a spacer residue, it is more preferred that the carboxy group of the polysaccharide and the spacer residue are linked through an amide bond.

In the formula 2, Y is a spacer residue or an ester bond, and Z is a diclofenac residue. In a preferred embodiment, the polysaccharide derivative has a structure in which part of the carboxy groups of the polysaccharide and the diclofenac residue Z are linked through the spacer residue.

When Y is an ester bond (X and Z are directly bonded to each other), X and Z are linked by an ester bond between the hydroxy group of the polysaccharide and the carboxy group of Z.

When Y is a spacer residue, the bond between Y and Z is selected from the group consisting of an ester, a thioester, and an amide. When Y is a spacer residue, it is preferred that the bond between Y and Z is an ester.

In a preferred embodiment, between X and A is an amide bond of the carboxy group of the polysaccharide and the substituent A; Y is a spacer residue; and the bond between Y and Z is an ester.

The spacer residue is not particularly limited, but a divalent linking group selected from the group consisting of a C₁₋₆ alkylene group, an amino acid residue, and a polypeptide chain can be exemplified. As the C₁₋₆ alkylene group, more specifically, for example, a methylene group, an ethylene group, a trimethylene group, an isopropylene group, etc. can be exemplified. As the amino acid residue, more specifically, for example, a glycine residue, a β-alanine residue, a γ-aminobutyric acid residue, etc. can be exemplified. The polypeptide chain can be, for example, a polypeptide chain having 2 to 12 amino acid residues. Among the above-mentioned spacer residues, a C₁₋₆ alkylene group is preferably used, and an ethylene group, a trimethylene group, and an isopropylene group are more preferably used.

As a compound (spacer compound) to be used as the spacer residue, a compound having at least one first functional group that binds to the carboxy group and/or the hydroxy group of the polysaccharide and at least one second functional group that binds to diclofenac may be appropriately selected according to the bond form between the polysaccharide and diclofenac.

For example, when the spacer residue is introduced by forming an amide bond with the carboxy group of the polysaccharide, a spacer compound having an amino group can be selected. When the spacer residue is introduced by forming an ester bond with the carboxy group of the polysaccharide, a spacer compound having a hydroxy group can be selected. When the spacer residue is introduced by forming a thioester bond with the carboxy group of the polysaccharide, a spacer compound having a mercapto group can be selected. When the spacer residue is introduced by forming an ester bond with the hydroxy group of the polysaccharide, a spacer compound having a carboxy group can be selected. As the spacer compound, from the viewpoint of ease of introduction into the polysaccharide and stability in the living body, the bond form between the polysaccharide residue and the spacer residue is preferably an amide bond.

Further, for example, when the spacer residue is introduced by forming an ester bond with the carboxy group of diclofenac, a spacer compound having a hydroxy group can be selected. When the spacer residue is introduced by forming an amide bond with the carboxy group of diclofenac, a spacer compound having an amino group can be selected. When the spacer residue is introduced by forming a thioester bond with the carboxy group of diclofenac, a spacer compound having a mercapto group can be selected. From the viewpoint that diclofenac is released by biodegradation, the bond form between the spacer residue and the diclofenac residue is preferably an ester bond.

The spacer compound can be appropriately selected according to the bond form between the polysaccharide and diclofenac as described above, but examples thereof include a C₁₋₆ diaminoalkane, an aminoalkyl alcohol having 1 to 6 carbon atoms, an amino acid, and a polypeptide. The amino acid may be a natural or unnatural amino acid, and is not particularly limited, but examples thereof include glycine, β-alanine, and γ-aminobutyric acid.

As the diclofenac used in the synthesis of the polysaccharide derivative, free diclofenac and salts such as diclofenac sodium and diclofenac potassium can be exemplified.

A method for introducing the spacer residue and the diclofenac residue into the polysaccharide is not particularly limited. That is, the diclofenac residue may be introduced into the polysaccharide into which the spacer residue has been introduced, or diclofenac into which the spacer residue has previously been introduced may be reacted with the polysaccharide.

A method for binding the polysaccharide, diclofenac, and the spacer compound to one another is not particularly limited. For example, a conventional method generally used as a means for performing the binding reaction can be used as long as it is a method capable of forming an ester, a thioester, an amide, or the like, and also the reaction conditions can be appropriately determined and selected by a person skilled in the art.

As a method for achieving binding of the spacer compound or the spacer-bound diclofenac and the carboxy group or the hydroxy group of the polysaccharide, for example, a method using a water-soluble condensing agent such as water-soluble carbodiimide (for example, 1-ethyl-3-(3-dimethylaminopropyl)-carbodiimide hydrochloride (EDCI⋅HCl), 1-ethyl-3-(3-dimethylaminopropyl) carbodiimide methiodide, or the like), a method using a condensing aid such as N-hydroxysuccinimide (HOSu) or N-hydroxybenzotriazole (HOBt) and the above-mentioned condensation agent, an active ester method, an acid anhydride method, etc. are exemplified.

The formulation is characterized by being for use in promotion of hyaluronic acid synthesis in a subject. The “promotion of hyaluronic acid synthesis” in the description can include an increase in the molecular weight of hyaluronic acid to be synthesized in a subject and/or improvement of the production rate of hyaluronic acid. The increase in the molecular weight of hyaluronic acid can be an increase in the production amount of hyaluronic acid having a molecular weight of 2,000,000 or more.

In an embodiment, the formulation contains not less than 0.01 wt % and not more than 80 wt % of the polysaccharide derivative or a salt thereof. In another embodiment, the formulation contains not less than 0.1 wt % and not more than 10 wt % of the polysaccharide derivative or a salt thereof.

The formulation can contain a carrier in addition to the polysaccharide derivative or a salt thereof. Preferred examples of the carrier include aqueous solvents such as sterile purified water, phosphate buffered saline (PBS), and physiological saline. In an embodiment, the formulation is prepared by mixing the carrier and the polysaccharide derivative. If necessary, an additive such as a buffer may be added to the formulation. In addition, the formulation may be subjected to a treatment for dust removal, microbial removal, sterilization, or the like by, for example, filtration through a filter or the like after mixing the respective components. In an embodiment, the formulation is in the form of a powder. In another embodiment, the formulation is in the form of a solution or a gel.

In a preferred embodiment, the formulation is for use in a subject that produces hyaluronic acid having a molecular weight peak of 2,000,000 or less. Note that the “molecular weight peak” is a peak protruding upward in fraction numbers 1 to 17 when hyaluronic acid in a sample is separated by the following method using high-performance liquid chromatography (HPLC), and fractions are collected every 0.5 minutes using a fraction collector.

### (Measurement Method for Molecular Weight Peak)

Mobile phase: a solution of 5 mmol/L phosphate buffer solution (pH 6.0) with 0.82% (w/v) NaCl:acetonitrile=2:1 (v:v)

Flow rate: 0.5 mL/min

Column: OH pak SB-807 HQ column, Shodex (registered trademark), column temperature: 35° C.

Injection volume: 10 μL

The sample can be, for example, a culture medium in which synovial cells were cultured or a joint fluid, or the like.

One aspect of the present invention relates to a kit including at least the following (A) and (B):

(A) a polysaccharide derivative represented by the above formula 1 or a salt thereof; and

(B) an instruction manual or a label indicating that the kit is for use in promotion of hyaluronic acid synthesis.

In an embodiment, the polysaccharide derivative of the formula 1 or a salt thereof included in the kit is filled in a container such as a vial or a reagent bottle. In a certain embodiment, the formulation filled in a container can be provided in a sterile state.

The above (B) may be an instruction manual or a label indicating that the polysaccharide derivative represented by the above formula 1 or a salt thereof promotes hyaluronic acid synthesis.

One aspect of the present invention relates to a method for promoting hyaluronic acid synthesis including bringing an effective amount of the polysaccharide derivative represented by the formula 1 or a salt thereof into contact with hyaluronic acid-producing cells. The method for bringing the polysaccharide derivative or a salt thereof into contact with hyaluronic acid-producing cells is not particularly limited. In an embodiment, the contact can be carried out by culturing hyaluronic acid-producing cells in a culture medium containing the polysaccharide derivative represented by the formula 1 or a salt thereof.

The “hyaluronic acid-producing cells” in the description are not particularly limited as long as they are animal cells that produce hyaluronic acid, but for example, synovial cells, chondrocytes, fibroblasts, keratinocytes, smooth muscle cells, oral mucosal cells, vascular endothelial cells, mammary epithelial cells, etc. can be exemplified. Among these, synovial cells are preferably used.

One aspect of the present invention relates to use of the polysaccharide derivative represented by the formula 1 or a salt thereof as a method for promoting hyaluronic acid synthesis.

One aspect of the present invention relates to a method for evaluating the responsiveness of hyaluronic acid-producing cells to the polysaccharide derivative represented by the formula 1 or a salt thereof, including (1) culturing the hyaluronic acid-producing cells in a culture medium containing the polysaccharide derivative of the formula 1 or a salt thereof, and (2) measuring the molecular weight and/or the content of hyaluronic acid in the culture medium. The measurement of the molecular weight and/or the content of hyaluronic acid in the culture medium may be performed by, for example, the method described in Examples, or may be performed using a commercially available kit for quantitative determination of hyaluronic acid or reagent for measurement of hyaluronic acid, or the like.

The method for evaluating the responsiveness of hyaluronic acid-producing cells described above may include, as a step (3), detecting the presence of responsiveness of the hyaluronic acid-producing cells to the polysaccharide derivative of the formula 1 or a salt thereof using as an index an increase in the molecular weight and/or the content of hyaluronic acid measured in the step (2). In an embodiment, the increase in the molecular weight and/or the content of hyaluronic acid can be an increase with respect to the molecular weight and/or the content of hyaluronic acid in a culture medium after culturing the hyaluronic acid-producing cells in a culture medium not containing the polysaccharide derivative of the formula 1 or a salt thereof. In another embodiment, the increase in the molecular weight and/or the content of hyaluronic acid can be an increase with respect to the molecular weight and/or the content of hyaluronic acid in a culture medium before culturing in the step (1).

One aspect of the present invention relates to a method for evaluating the responsiveness of the polysaccharide derivative represented by the formula 1 or a salt thereof to hyaluronic acid-producing cells, including (1) culturing the hyaluronic acid-producing cells in a culture medium containing the polysaccharide derivative of the formula 1 or a salt thereof, and (2) measuring the molecular weight and/or the content of hyaluronic acid in the culture medium. The method for evaluating the responsiveness of the polysaccharide derivative represented by the formula 1 or a salt thereof may include, as a step (3), detecting the presence of responsiveness of the hyaluronic acid derivative or a salt thereof to the hyaluronic acid-producing cells using as an index an increase in the molecular weight and/or the content of hyaluronic acid measured in the step (2).

One aspect of the present invention relates to a method for producing hyaluronic acid including (1′) culturing hyaluronic acid-producing cells in a culture medium containing the polysaccharide derivative represented by the formula 1 or a salt thereof, and (2′) collecting hyaluronic acid from the culture medium. By culturing the hyaluronic acid-producing cells in a culture medium containing the polysaccharide derivative represented by the formula 1 or a salt thereof, HA having a large molecular weight can be obtained, or the production amount of HA can be increased.

The collection of hyaluronic acid from the culture medium can be carried out by a person skilled in the art using a conventionally known method such as salting out, ammonium sulfate fractionation, centrifugation, dialysis, ultrafiltration, adsorption chromatography, ion exchange chromatography, hydrophobic chromatography, reverse-phase chromatography, gel permeation chromatography, affinity chromatography, or electrophoresis, or a combination thereof. The collected hyaluronic acid may be dried by a conventionally known means as needed.

One aspect of the present invention relates to use of the polysaccharide derivative represented by the formula 1 or a salt thereof in the production of a hyaluronic acid synthesis promoter. The polysaccharide derivative represented by the formula 1 or a salt thereof can be used as a hyaluronic acid synthesis promoter. The hyaluronic acid synthesis promoter, in other words, may be an agent having an action of promoting hyaluronic acid synthesis. Therefore, the polysaccharide derivative represented by the formula 1 or a salt thereof can also be used for producing a formulation for promoting hyaluronic acid synthesis.

### EMBODIMENTS

Preferred embodiments of the present invention will be illustrated below.

[1] A formulation including a polysaccharide derivative or a salt thereof, wherein

the polysaccharide derivative is represented by the following formula 1,

the formulation being for use in promotion of hyaluronic acid synthesis:

[Chem. 3]

XA)ₙ formula 1

wherein X is a residue derived from a polysaccharide having at least one of a carboxy group and a hydroxy group; A is a substituent; n is an introduction ratio of the substituent A and is not less than 1 mol % and not more than 80 mol %; between X and A is a bond of the carboxy group or the hydroxy group and the substituent A, and the bond is selected from the group consisting of an ester, a thioester, and an amide; and the substituent A is represented by the following formula 2:

[Chem. 4]

*—Y—Z formula 2

wherein Y is a spacer residue or an ester bond; Z is a diclofenac residue; when Y is a spacer residue, the bond between Y and Z is selected from the group consisting of an ester, a thioester, and an amide; and is a binding site to X.

[2] The formulation according to [1], wherein the polysaccharide is selected from the group consisting of hyaluronic acid, chondroitin, chondroitin sulfate, heparin, heparan sulfate, and carboxy C₁₋₄ alkyldextran.

[3] The formulation according to [1] or [2], wherein the spacer residue is selected from the group consisting of a C₁₋₆ alkylene group, an amino acid residue, and a polypeptide chain.

[4] The formulation according to any one of [1] to [3], wherein the promotion of hyaluronic acid synthesis is an increase in the molecular weight of hyaluronic acid to be synthesized in a subject for which the formulation is used.

[5] The formulation according to any one of [1] to [4], wherein the average molecular weight of the polysaccharide is not less than 10,000 and not more than 5,000,000.

[6] A kit, containing at least the following (A) and (B):

(A) a polysaccharide derivative represented by the above formula 1 or a salt thereof; and

(B) an instruction manual or a label indicating that the kit is for use in promotion of hyaluronic acid synthesis.

[7] A method for promoting hyaluronic acid synthesis, comprising a step of bringing a polysaccharide derivative represented by the above formula 1 or a salt thereof into contact with hyaluronic acid-producing cells.

[8] The method for promoting hyaluronic acid synthesis according to [7], wherein the polysaccharide is selected from the group consisting of hyaluronic acid, chondroitin, chondroitin sulfate, heparin, heparan sulfate, and carboxy C₁₋₄ alkyldextran.

[9] The method for promoting hyaluronic acid synthesis according to [7] or [8], wherein the spacer residue in the formula 1 is selected from the group consisting of a C₁₋₆ alkylene group, an amino acid residue, and a polypeptide chain.

[10] The method for promoting hyaluronic acid synthesis according to any one of [7] to [9], wherein the promotion of hyaluronic acid synthesis is an increase in the molecular weight of hyaluronic acid to be synthesized in the hyaluronic acid-producing cells.

[11] The method for promoting hyaluronic acid synthesis according to any one of [7] to [10], wherein the average molecular weight of the polysaccharide is not less than 10,000 and not less than 5,000,000.

[12] The method for promoting hyaluronic acid synthesis according to any one of [7] to [11], wherein the hyaluronic acid-producing cells are selected from the group consisting of synovial cells, chondrocytes, fibroblasts, keratinocytes, smooth muscle cells, oral mucosal cells, vascular endothelial cells, and mammary epithelial cells.

[13] A method for evaluating the responsiveness of hyaluronic acid-producing cells to a polysaccharide derivative represented by the above formula 1 or a salt thereof, including:

(1) a step of culturing the hyaluronic acid-producing cells in a culture medium containing the polysaccharide derivative or a salt thereof; and

(2) a step of measuring the molecular weight and/or the content of hyaluronic acid in the culture medium.

[14] The method according to [13], further including: (3) a step of detecting the presence of responsiveness of the hyaluronic acid-producing cells to the polysaccharide derivative or a salt thereof using an increase in the molecular weight and/or the content of hyaluronic acid as an index.

[15] A method for producing hyaluronic acid, including:

(1′) a step of culturing hyaluronic acid-producing cells in a culture medium containing a polysaccharide derivative represented by the above formula 1 or a salt thereof; and

(2′) a step of collecting hyaluronic acid from the culture medium.

[16] A kit, including at least the following (A) and (B):

(A) a polysaccharide derivative represented by the above formula 1 or a salt thereof; and

(B) an instruction manual or a label indicating that the polysaccharide derivative represented by the above formula 1 or a salt thereof promotes hyaluronic acid synthesis.

[17] A method for evaluating the responsiveness of a polysaccharide derivative represented by the above formula 1 or a salt thereof to hyaluronic acid-producing cells, including:

(1) a step of culturing the hyaluronic acid-producing cells in a culture medium containing the polysaccharide derivative or a salt thereof; and

(2) a step of measuring the molecular weight and/or the content of hyaluronic acid in the culture medium.

[18] The method according to [17], further including (3) a step of detecting the presence of responsiveness of the polysaccharide derivative or a salt thereof to the hyaluronic acid-producing cells using an increase in the molecular weight and/or the content of hyaluronic acid as an index.

[19] Use of a polysaccharide derivative represented by the above formula 1 or a salt thereof as a method for promoting hyaluronic acid synthesis.

[20] The use according to [19], wherein the method for promoting hyaluronic acid synthesis includes a step of bringing the polysaccharide derivative represented by the formula 1 or a salt thereof into contact with hyaluronic acid-producing cells.

[21] Use of a polysaccharide derivative represented by the above formula 1 or a salt thereof as a hyaluronic acid synthesis promoter in a method for evaluating the response of hyaluronic acid-producing cells.

[22] The use according to [21], wherein the method for evaluating the response of hyaluronic acid-producing cells includes the following steps:

(1) a step of culturing the hyaluronic acid-producing cells in a culture medium containing the polysaccharide derivative or a salt thereof; and

(2) a step of measuring the molecular weight and/or the content of hyaluronic acid in the culture medium.

[23] The use according to [22], wherein the method for evaluating the response of hyaluronic acid-producing cells further includes (3) a step of detecting the presence of responsiveness of the hyaluronic acid-producing cells to the polysaccharide derivative or a salt thereof using an increase in the molecular weight and/or the content of hyaluronic acid as an index.

[24] Use of hyaluronic acid-producing cells as a hyaluronic acid synthesis promoter in a method for evaluating the responsiveness of a polysaccharide derivative represented by the above formula 1 or a salt thereof.

[25] The use according to [24], wherein the evaluation of the responsiveness of the polysaccharide derivative represented by the above formula 1 or a salt thereof includes the following steps:

(1) a step of culturing the hyaluronic acid-producing cells in a culture medium containing the polysaccharide derivative or a salt thereof; and

(2) a step of measuring the molecular weight and/or the content of hyaluronic acid in the culture medium.

[26] The use according to [25], wherein the evaluation of the responsiveness of the polysaccharide derivative represented by the above formula 1 or a salt thereof further includes (3) a step of detecting the presence of responsiveness of the polysaccharide derivative or a salt thereof to the hyaluronic acid-producing cells using an increase in the molecular weight and/or the content of hyaluronic acid as an index.

### EXAMPLES

Hereinafter, preferred embodiments of the present invention will be described in more detail using Examples, however, the technical scope of the present invention is not limited to the following Examples. Further, unless otherwise noted, operations and measurements of physical properties and the like were performed under the conditions of room temperature (not less than 20° C. and not more than 25° C.) and a relative humidity of not less than 40% RH and not more than 50% RH.

### Example 1

The action of promoting hyaluronic acid synthesis of a compound represented by the formula 1 was verified in a synovial cell culture system.

### Synthesis Example

Aminoethanol-diclofenac-introduced sodium hyaluronate (compound 1) was synthesized by the following procedure.

2.155 g (10.5 mmol) of 2-bromoethylamine hydrobromide was dissolved in 20 mL of dichloromethane, and 1.463 mL (10.5 mmol) of triethylamine was added under ice-cooling, and further 5 mL of a dichloromethane solution of 2.299 g (10.5 mmol) of di-tert-butyl-dicarbonate (Boc₂O) was added thereto, followed by stirring. After stirring at room temperature for 90 minutes, ethyl acetate was added thereto, and the resulting mixture was washed sequentially with a 5 wt % citric acid aqueous solution, water, and saturated brine. After dehydration with sodium sulfate, the solvent was distilled off under reduced pressure, whereby Boc-aminoethyl bromide was obtained.

5 mL of a dimethylformamide (DMF) solution of 2.287 g (10.2 mmol) of the thus obtained Boc-aminoethyl bromide was ice-cooled, and 6 mL of a DMF solution of 3.255 g (10.2 mmol) of diclofenac sodium (Wako Pure Chemical Industries, Ltd.) was added thereto, followed by stirring overnight at room temperature. Stirring was performed at 60° C. for 11 hours, and then stirring was performed overnight at room temperature. Ethyl acetate was added thereto, and the resulting mixture was washed sequentially with a 5 wt % sodium hydrogen carbonate aqueous solution, water, and saturated brine. After dehydration with sodium sulfate, ethyl acetate was distilled off under reduced pressure. The residue was purified by silica gel column chromatography (toluene:ethyl acetate=20:1 (v/v), 0.5 vol % triethylamine), whereby Boc-aminoethanol-diclofenac was obtained.

2.108 g (4.80 mmol) of the thus obtained Boc-aminoethanol-diclofenac was dissolved in 5 mL of dichloromethane, and 20 mL of 4 M hydrochloric acid/ethyl acetate was added under ice cooling, followed by stirring for 2.5 hours. Diethyl ether and hexane were added thereto to cause precipitation, and the precipitate was dried under reduced pressure. As a result, aminoethanol-diclofenac hydrochloride was obtained. The structure was identified by ¹H-NMR. ¹H-NMR (500 MHz, CDCl₃) δ (ppm)=3.18 (2H, t, NH₂CH₂CH₂O—), 3.94 (2H, s, Ph-CH₂—CO), 4.37 (2H, t, NH₂CH₂CH₂O—), 6.47-7.31 (8H, m, Aromatic H, NH)

After 500 mg (1.25 mmol/disaccharide unit) of hyaluronic acid (weight average molecular weight: 800,000) was dissolved in water (56.3 mL)/dioxane (56.3 mL), hydroxysuccinimide (1 mmol)/water (0.5 mL), water-soluble carbodiimide hydrochloride (WSCI⋅HCl) (0.5 mmol)/water (0.5 mL), aminoethanol-diclofenac hydrochloride (0.5 mmol) obtained above/(water:dioxane=1:1 (v/v) (5 mL)) were sequentially added thereto, followed by stirring for a whole day and night. To the reaction solution, 7.5 mL of a 5 wt % sodium hydrogen carbonate aqueous solution was added, followed by stirring for about 4 hours. The reaction solution was neutralized by adding 215 μL of a 50% (v/v) acetic acid aqueous solution, and then 2.5 g of sodium chloride was added thereto, followed by stirring. 400 ml of ethanol was added to cause precipitation, and the precipitate was washed twice with 85% (v/v) ethanol aqueous solution, twice with ethanol and twice with diethyl ether, and then dried under reduced pressure overnight at room temperature, whereby aminoethanol-diclofenac-introduced sodium hyaluronate (compound 1) was obtained. The introduction ratio of diclofenac measured by a spectrophotometer was 18 mol %.

### (Test Substance)

Compound 1 or sodium hyaluronate (HA) (ARTZ Dispo (registered trademark) (manufactured by Seikagaku Corporation)) was mixed in a solution containing a phosphate buffer solution (GIBCO) and an α-MEM (GIBCO) concentrated medium. Further, each reagent was added and mixed in the resulting solution so that the final concentrations were as follows: 10% (v/v) fetal bovine serum (hereinafter FBS (MP Biomedicals)), 10 ng/mL recombinant human IL-1β/IL-1F2 (hereinafter IL-1β (R & D Systems)), 1% (w/v) penicillin/streptomycin (GIBCO), and 370 kBq/mL [³H] glucosamine (Perkin Elmer). As a result, the following five solutions were obtained as test substances.

(1) a control solution (not containing the compound 1 or HA)

(2) a 0.1% (w/v %) compound 1 solution

(3) a 0.1% (w/v %) sodium hyaluronate (HA) solution

(4) a 0.01% (w/v %) compound 1 solution

(5) a 0.01% (w/v %) sodium hyaluronate (HA) solution

### (Test Method)

(1) Cell Culture, Addition of Test Substance, and Collection of Culture Supernatant

Human synovial cells derived from a patient with rheumatism (HFLS-RA, CELL APPLICATIONS, INC.) were subcultured and grown in a 175-cm² flask. In the cell culture, Basal medium (containing 10% (v/v) Growth supplement and 1% (w/v) penicillin/streptomycin) (manufactured by Cell Applications, Inc.) was used. Thereafter, the cells were seeded in a 6-well plate at 3.0×10⁵ cells/2 mL/well and cultured for about 24 hours until confluence was reached. In the cell culture, α-MEM medium (containing 10% (v/v) FBS and 1% (w/v) penicillin/streptomycin) was used. After removing the culture supernatant, 2 mL of the test substance was added to the cells, and the cells were further cultured for 48 hours. The culture was performed at 37° C. in a CO₂ incubator (5% (v/v) CO₂). After completion of the culture, the culture supernatant was collected and cryopreserved in an ultra-low temperature freezer until measurement.

(2) Fractionation of Culture Supernatant and Measurement of Radioactivity

Since glucosamine is a constituent monosaccharide of hyaluronic acid, in hyaluronic acid newly synthesized in the cells after addition of the test substance, [³H] glucosamine is incorporated. Therefore, hyaluronic acid in the collected culture supernatant was separated according to molecular weight using HPLC, and fractions were collected every 0.5 minutes using a fraction collector. To each of the collected fractions, 0.5 mL of a scintillation fluid was added and mixed. Thereafter, the radioactivity (dpm, disintegrations per minute) of each fraction was measured using a scintillation counter, and the radioactivity (the amount of [³H] glucosamine incorporated into newly produced hyaluronic acid) was evaluated. The HPLC conditions are as follows.

Mobile phase: a solution of 5 mmol/L phosphate buffer solution (pH 6.0) with 0.82% (w/v) NaCl:acetonitrile=2:1 (v:v)

Flow rate: 0.5 mL/min

Column: OH pak SB-805 HQ column (Shodex (registered trademark)), column temperature: 35° C.

Injection volume: 10 μL

Scintillation counter conditions

³H, dpm, 3 min

Scintillation fluid: Ultima-Flo™ M, cocktail for flow scintillation analyzer

The hyaluronic acid standard solution was separated by HPLC, and UV absorption at a wavelength of 210 nm was measured. Fraction No. in which the peak top of the hyaluronic acid standard solution of each molecular weight is collected was calculated. As the hyaluronic acid standard solutions, Select-HATM 500 k (average molecular weight: 528,000), Select-HATM 1,000 k (average molecular weight: 1,076,000), and Select-HATM 2,500 k (average molecular weight: 2,420,000) were used.

### (Results)

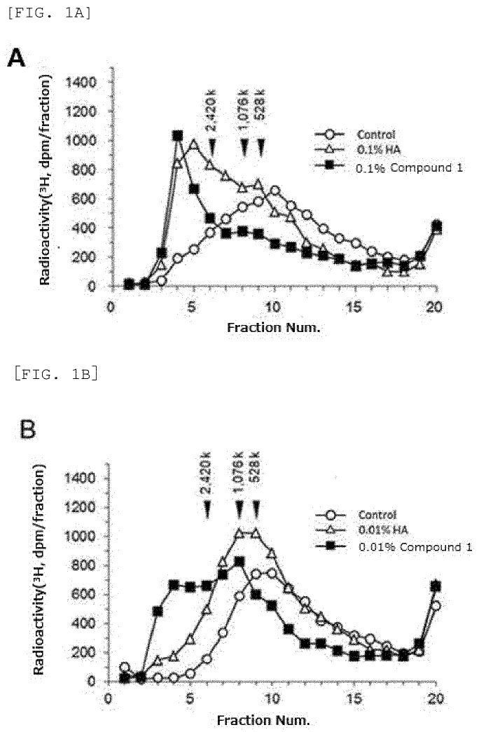

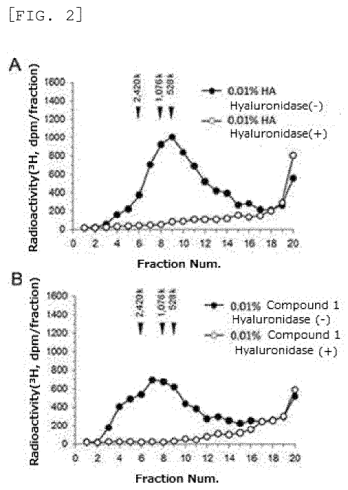

The results are shown in FIGS. 1A and 1B.

### FIG. 1A

1) a control group (n=2)

2) a 0.1% (w/v %) sodium hyaluronate (HA) group (n=1)

3) a 0.1% (w/v %) compound 1 group (n=1)

### FIG. 1B

1) a control group (n=1)

2) a 0.01% (w/v %) sodium hyaluronate (HA) group (n=1)

3) a 0.01% (w/v %) compound 1 group (n=1)

In FIGS. 1A and 1B, the results are shown as average values (the number of cases in each group=1 to 2).

It was shown in FIGS. 1A and 1B that hyaluronic acid having a peak in the vicinity of 500,000 was produced in the control group. In addition, it was also shown that in the HA group, hyaluronic acid having a peak in the vicinity of 500,000 to 2,400,000 was produced, and in the compound 1 group, hyaluronic acid having a high molecular weight exceeding the molecular weight of hyaluronic acid produced in the HA group was produced in a concentration-dependent manner. The hyaluronic acid produced in the compound 1 group had a higher molecular weight than that in the HA group at any concentration.

From the above results, it was revealed that the compound 1 has an action of promoting the synthesis of high molecular weight hyaluronic acid on human synovial cells derived from a patient with rheumatism. The action exceeded that of sodium hyaluronate and was also observed even at a concentration of 0.01% (w/v).

### Example 2

It was verified that the substance whose synthesis was promoted by the action of the compound 1 is hyaluronic acid by confirming the presence or absence of degradation by a hyaluronic acid-degrading enzyme (hyaluronidase).

### (Test Substance)

As test substances, the following two solutions were prepared in the same manner as in Example 1.

(1) a 0.01% (w/v %) sodium hyaluronate (HA) solution

(2) a 0.01% (w/v %) compound 1 solution

### (Test Method)

(1) Cell Culture, Addition of Test Substance, and Collection of Culture Supernatant

The same procedure as in Example 1 was performed.

(2) Hyaluronidase Treatment

The culture supernatant (90 μL) collected after adding the 0.01% HA solution and 10 μL of 100 TRU/mL hyaluronidase (or water for injection) were mixed and reacted overnight at 37° C., thereby performing a hyaluronidase treatment. The culture supernatant (90 μL) collected after adding the 0.01% compound 1 solution and 30 μL of 100 TRU/mL hyaluronidase (or water for injection) were mixed and reacted at 60° C. for 3 hours, thereby performing a hyaluronidase treatment. The hyaluronidase (derived from an actinomycetes) was purchased from Seikagaku Biobusiness Corporation.

(3) Fractionation of Culture Supernatant and Measurement of Radioactivity

The same procedure as in Example 1 was performed.

### (Results)

The results are shown in FIG. 2.

FIG. 2A (each n=1)

1) a 0.01% HA (hyaluronidase (−)) group

2) a 0.01% HA (hyaluronidase (+)) group

FIG. 2B (each n=1)

1) a 0.01% compound 1 (hyaluronidase (−)) group

2) a 0.01% compound 1 (hyaluronidase (+)) group

The production of a high molecular weight radioactive substance was confirmed in both the 0.01% HA (hyaluronidase (−)) group and the 0.01% compound 1 (hyaluronidase (−)) group. The radioactive substance in the 0.01% compound 1 (hyaluronidase (−)) group had a higher molecular weight than the radioactive substance in the 0.01% HA (hyaluronidase (−)) group.

On the other hand, in both the 0.01% HA (hyaluronidase (+)) group and the 0.01% compound 1 (hyaluronidase (+)) group, disappearance of the peak of the high molecular weight radioactive substance was confirmed.

From the above results, it was confirmed that the substance whose molecular weight was increased by the addition of HA or the compound 1 was hyaluronic acid.

### Example 3

A change in the action of promoting hyaluronic acid synthesis of the compound 1 over time was verified.

### (Test Substance)

As test substances, the following three solutions were prepared in the same manner as in Example 1.

(1) a control solution

(2) a 0.1% (w/v %) compound 1 solution

(3) a 0.1% (w/v %) sodium hyaluronate (HA) solution

### (Test Method)

The same procedure as in Example 1 was performed. Note that the culture supernatant was collected at respective time points (8, 24, 36, 48, and 72 hours) after adding the test substance to the cells.

### (Results)

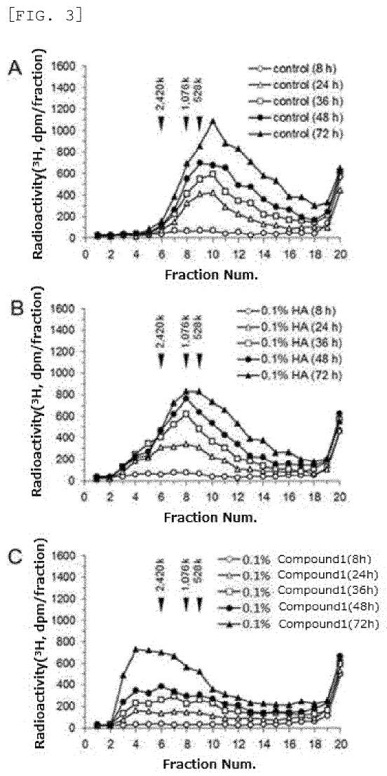

The results are shown in FIG. 3.

FIG. 3A: control (8, 24, 36, 48, and 72 hours) groups (each n=1)
FIG. 3B: 0.1% HA (8, 24, 36, 48, and 72 hours) groups (each n=1)
FIG. 3C: 0.1% compound 1 (8, 24, 36, 48, and 72 hours) groups (each n=1)

It was confirmed that in all the control groups, the HA groups, and the compound 1 groups, hyaluronic acid is produced, and the content of hyaluronic acid increases over time. In the control groups, hyaluronic acid having a peak in the vicinity of 500,000 was produced. In the HA groups, hyaluronic acid having a peak in the vicinity of 1,000,000 was produced. In the compound 1 groups, hyaluronic acid having a higher molecular weight than that in the HA groups was produced.

From the above results, both the compound 1 and sodium hyaluronate induced the production of high molecular weight hyaluronic acid, and increased the high molecular weight hyaluronic acid in the culture medium over time. In addition, the molecular weight of hyaluronic acid in the compound 1 group was larger than that in the HA group. With respect to the molecular weight, a fluctuation in the peak over time was not observed in any group. The compound 1 always had an action of producing endogenous high molecular weight hyaluronic acid during the measurement period.

### Example 4

The action of promoting hyaluronic acid synthesis of the compound 1 was compared with that of sodium hyaluronate and diclofenac sodium (DF-Na) that are the constituent components of the compound 1.

### (Test Substance)

A control solution, a 0.01% (w/v %) compound 1 solution, and a 0.01% (w/v %) sodium hyaluronate (HA) solution were prepared in the same manner as in Example 1. DF-Na (0.014 μg/mL, 0.14 μg/mL, 1.4 μg/mL, and 14 μg/mL) solutions were prepared by dissolving DF-Na in the control solution. Further, a HA+DF-Na mixed liquid was obtained by dissolving DF-Na (1.4 μg/mL) in the 0.01% (w/v %) sodium hyaluronate (HA) solution.

(1) a control solution

(2) a 0.01% (w/v %) compound 1 solution

(3) a 0.01% (w/v %) sodium hyaluronate (HA) solution

(4) DF-Na (0.014, 0.14, 1.4, and 14 μg/mL) solutions

(5) a 0.01% (w/v %) sodium hyaluronate (HA)+DF-Na (1.4 μg/mL) mixed liquid

### (Test Method)

The same procedure as in Example 1 was performed.

### (Results)

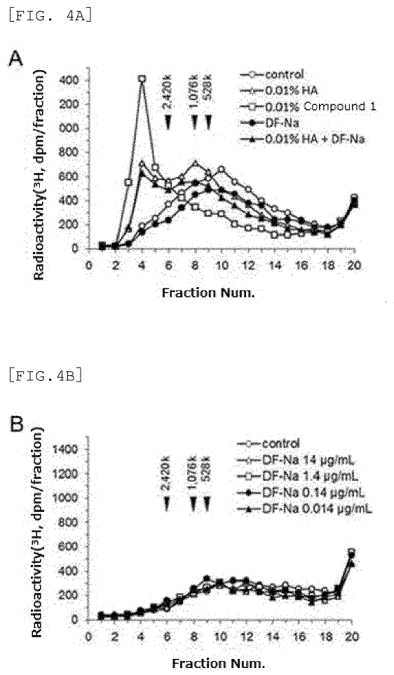

The results are shown in FIGS. 4A and 4B.

FIG. 4A (each n=2)

1) a control group

2) a 0.01% compound 1 group

3) a 0.01% HA group

4) a DF-Na (1.4 μg/mL) group

5) a 0.01% HA+DF-Na (1.4 μg/mL) group

### FIG. 4B

1) a control group (n=1)

2) DF-Na (0.014, 0.14, 1.4, and 14 μg/mL) groups (each n=1)

Note that the concentration of DF-Na (14 μg/mL) corresponds to the concentration of DF-Na contained in the 0.01% compound 1.

In the control group, the production of hyaluronic acid having a peak in the vicinity of 500,000 was confirmed, and in the HA group, the production of hyaluronic acid having a peak in the vicinity of 1,000,000 was confirmed (FIG. 4A). In the compound 1 group, the production of high molecular weight hyaluronic acid was more prominent than in the HA group.

In the HA+DF-Na mixed liquid group, the production of hyaluronic acid having a peak in the vicinity of 1,000,000 was confirmed in the same manner as in the HA group (FIG. 4A).

In the DF-Na group, the production of hyaluronic acid having a peak in the vicinity of 500,000 was confirmed in the same manner as in the control group (FIG. 4A).

In the DF-Na group and the HA+DF-Na mixed liquid group, an action of producing high molecular weight hyaluronic acid observed in the compound 1 group was not observed (FIG. 4A). In addition, in DF-Na, an action of promoting hyaluronic acid synthesis was not observed at any concentration from 0.014 to 14 μg/mL (FIG. 4B).

From the above results, an action of producing high molecular weight hyaluronic acid observed by the administration of the compound 1 was not observed by the administration of sodium hyaluronate, DF-Na, or the HA+DF-Na mixed liquid. That is, the action of increasing the molecular weight of endogenous hyaluronic acid of the compound 1 is not observed by simple mixing of the constituent components of the compound 1, and therefore, it was revealed that it is an action characteristic of the polysaccharide derivative represented by the formula 1.

### Example 5

The action of promoting hyaluronic acid synthesis of the compound 1 was verified using human synovial cells. At that time, by using synovial cells derived from a plurality of humans, the effect of difference in patients was examined.

### (Test Substance)

As test substances, the following five solutions were prepared in the same manner as in Example 1.

(1) a control solution

(2) a 0.01% (w/v %) compound 1 solution

(3) a 0.01% (w/v %) sodium hyaluronate (HA) solution

(4) a 0.1% (w/v %) compound 1 solution

(5) a 0.1% (w/v %) sodium hyaluronate (HA) solution

### (Test Method)

(1) Cell Culture, Addition of Test Substance, and Collection of Culture Supernatant

The same procedure as in Example 1 was performed. Note that human synovial cells derived from three patients with osteoarthritis (HFLS-OA, CELL APPLICATIONS, INC.) and human synovial cells derived from three patients with rheumatism (HFLS-RA, CELL APPLICATIONS, INC.) were used. The cells derived from three patients with each disease (3 lots) were separately evaluated and the number of cases was set to 3.

(2) Fractionation of Culture Supernatant and Measurement of Radioactivity

The same procedure as in Example 1 was performed. Note that a column (OH pak SB-807 HQ column (Shodex (registered trademark)) having higher separability on the high molecular weight side than the HPLC column (OH pak SB-805 HQ column (Shodex (registered trademark)) was used.

(3) Measurement of Cell Count

After the culture supernatant was collected, the adhered cells were detached from each well. Thereafter, the cell count was measured using a trypan blue solution and a hemacytometer.

### (Results)

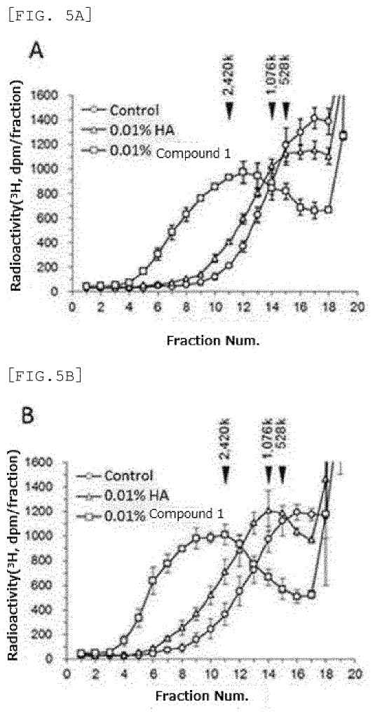

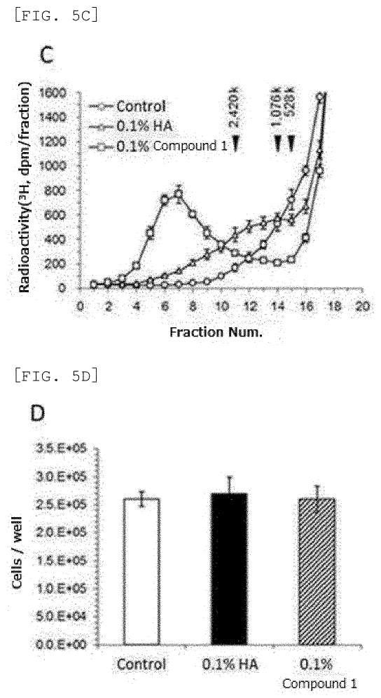

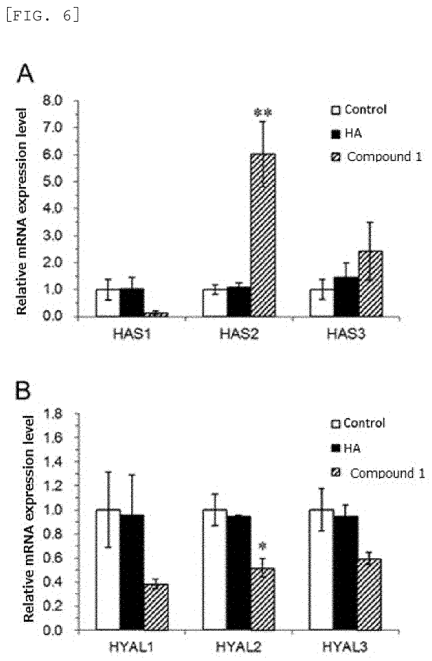

The results are shown in FIGS. 5A to 5D.

FIG. 5A (each n=3, synovial cells derived from three patients with rheumatism)

1) a control group

2) a 0.01% HA group

3) a 0.01% compound 1 group

FIG. 5B (each n=3, synovial cells derived from three patients with osteoarthritis)

1) a control group

2) a 0.01% HA group

3) a 0.01% compound 1 group

FIG. 5C (each n=3, synovial cells derived from three patients with osteoarthritis)

1) a control group

2) a 0.1% HA group

3) a 0.1% compound 1 group

FIG. 5D (each n=3, synovial cells derived from three patients with osteoarthritis)

1) a control group

2) a 0.1% HA group

3) a 0.1% compound 1 group

In each of FIGS. 5A to 5D, the results are shown as average values±standard error (each n=3). In FIG. 5A (synovial cells derived from patients with rheumatism) and FIG. 5B (synovial cells derived from patients with osteoarthritis), in the 0.01% compound 1 group, the production of high molecular weight hyaluronic acid having a peak in the vicinity of 2,400,000 was confirmed, and in the 0.01% HA group, the production of hyaluronic acid having a peak in the vicinity of 1,000,000 was confirmed. In FIG. 5C (synovial cells derived from patients with osteoarthritis), in the 0.1% compound 1 group, the production of hyaluronic acid having a high molecular weight more than 2,400,000 was confirmed. By using the column having high separability on the high molecular weight side, the molecular weight of high molecular weight hyaluronic acid produced by the administration of the compound 1 became clearer. Further, it was found that hyaluronic acid having a higher molecular weight is produced in a compound 1 concentration-dependent manner. In FIG. 5D (synovial cells derived from patients with osteoarthritis), a significant difference in the cell count was not observed in each group (p>0.05 in a parametric Tukey-type multiple comparison test), and there was no significant increase or decrease in the cell count by the addition of the 0.1% compound 1 and the addition of 0.1% HA.

As described above, the action of producing high molecular weight hyaluronic acid by the administration of the compound 1 was also observed in the synovial cells derived from any of the patients with rheumatism and the patients with osteoarthritis. It was revealed that the increase in the molecular weight of hyaluronic acid by the compound 1 has a small difference between samples. Further, the compound 1 had an action of inducing the production of hyaluronic acid having a higher molecular weight than sodium hyaluronate. In addition, it was revealed that the action of producing high molecular weight hyaluronic acid of the compound 1 is not attributed to an increase in the cell count, but to an increase in the molecular weight of hyaluronic acid to be synthesized in the cells.

### Example 6

The mechanism of action of promoting hyaluronic acid synthesis of the compound 1 was examined.

### (Test Substance)

Compound 1 or sodium hyaluronate (HA) (ARTZ Dispo (registered trademark) (manufactured by Seikagaku Corporation)) was mixed in a solution containing a phosphate buffer solution and an α-MEM concentrated medium. Further, each reagent was added and mixed in the resulting solution so that the final concentrations were as follows: 10% (v/v) fetal bovine serum (hereinafter FBS), 10 ng/mL recombinant human IL-1β/IL-1F2 (hereinafter IL-1β), and 1% (w/v) penicillin/streptomycin. As a result, the following three solutions were prepared as test substances.

(1) a control solution

(2) a 0.1% (w/v %) compound 1 solution

(3) a 0.1% (w/v %) sodium hyaluronate (HA) solution

### (Test Method)

(1) Cell Culture, and Addition of Test Substance

Human synovial cells derived from a patient with osteoarthritis (HFLS-OA, CELL APPLICATIONS, INC.) were subcultured and grown in a 175-cm² flask. In the cell culture, Basal medium (containing 10% (v/v) Growth supplement and 1% (w/v) penicillin/streptomycin) (manufactured by Cell Applications, Inc.) was used. Thereafter, the cells were seeded in a 6-well plate at 3.0×10⁵ cells/2 mL/well and cultured for about 24 hours until confluence was reached. In the cell culture, α-MEM medium (containing 10% (v/v) FBS and 1% (w/v) penicillin/streptomycin) was used. After removing the culture supernatant, 2 mL of the test substance was added to the wells, and the cells were further cultured for 48 hours. The culture was performed at 37° C. in a CO₂ incubator (5% (v/v) CO₂).

(2) Extraction of RNA in Cultured Cells and Preparation of cDNA Sample

After completion of the culture, RNA was extracted from the cells for each well using RNeasy (registered trademark) Plus Mini kit (QIAGEN). After measuring the RNA level using an ultra-micro spectrophotometer, the sample was cryopreserved in an ultra-low temperature freezer. By using the extracted RNA, a cDNA sample was prepared using Super Script (registered trademark) III First-Strand Synthesis System (Invitrogen).

(3) Calculation of Relative mRNA Level (Real-Time PCR)

The cDNA sample was subjected to real-time PCR using Premix ExTaq (Perfect Real Time) (Takara Bio, Inc.), the Ct values of target genes (HAS1, HAS2, HAS3, HYAL1, HYAL2, and HYAL3) and GAPDH were measured, and relative mRNA levels were calculated by the ΔΔCt method.

As the probe &primer, Taqman (registered trademark) Gene Expression Assay (Applied Biosystems) was used: HAS1 (ID: Hs00987418_m1), HAS2 (ID: Hs00193435_m1), HAS3 (ID: Hs00193436_m1), HYAL1 (ID: Hs00201046_m1), HYAL2 (ID: Hs01117343_g1), HYAL3 (ID: Hs00185910_m1), and GAPDH (ID: Hs03929097_g1).

### (Results)

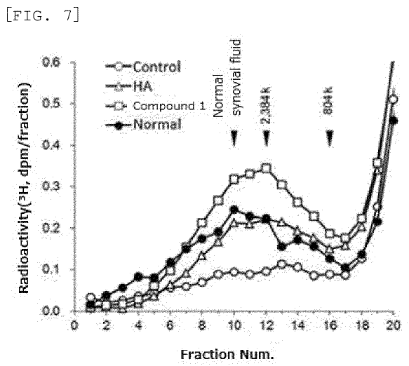

The results are shown in FIG. 6 (in each group, n=3, synovial cells derived from three patients with osteoarthritis).

FIG. 6A: Relative mRNA levels of HAS1, HAS2, and HAS3

1) a control group

2) a 0.1% HA group

3) a 0.1% compound 1 group

### FIG. 6B:

Relative mRNA levels of HYAL1, HYAL2, and HYAL3

1) a control group

2) a 0.1% HA group

3) a 0.1% compound 1 group

In FIG. 6, the results are shown as average values±standard error (the number of cases in each group=3). * and ** show p<0.05 and p<0.01 (vs control) in the Dunnett test, respectively.

It was found that the compound 1 significantly suppresses the expression of HYAL2 mRNA, and significantly promotes the expression of HAS2 mRNA. On the other hand, the effect of sodium hyaluronate on the expression of HYAL1, 2, 3 mRNA and HAS1, 2, 3 mRNA was not observed.

As described above, the compound 1 promoted the mRNA expression of HAS2 involved in the production of high molecular weight hyaluronic acid. From the above result, there is a high possibility that the promotion of the expression of HAS2 mRNA is involved in the mechanism of action of promoting hyaluronic acid synthesis of the compound 1. In addition, since the compound 1 suppressed the mRNA expression of HYAL2 involved in the degradation of high molecular weight hyaluronic acid, it was suggested that the suppression of the degradation of high molecular weight hyaluronic acid may also be involved in the mechanism of action.

### Example 7

The action of promoting hyaluronic acid synthesis of the compound 1 in a model rabbit with antigen-induced arthritis was verified.

### (Test Substance)

A 0.5% (w/v %) compound 1 solution was obtained by mixing respective solutions of a 1% (w/v) compound 1 solution (phosphate buffer solution), a 10% (w/v) ovalbumin solution (phosphate buffer solution) and [³H] glucosamine (phosphate buffer solution, 37 MBq/mL) at a ratio of 5:1:4 (v:v:v). In the preparation of a control solution, a phosphate buffer solution was used in place of the compound 1 solution. In the preparation of a sodium hyaluronate (HA) solution, ARTZ Dispo (registered trademark) (manufactured by Seikagaku Corporation) was used in place of the compound 1 solution. Further, a Normal solution not containing ovalbumin (an arthritis-inducing substance) was obtained by mixing respective solutions of a phosphate buffer solution and [³H] glucosamine (phosphate buffer solution, 37 MBq/mL) at a ratio of 3:2 (v:v). As test substances, the following substances were used.

(1) a control solution

(2) a 0.5% (w/v %) sodium hyaluronate (HA) solution

(3) a 0.5% (w/v %) compound 1 solution

(4) a Normal solution

### (Test Method)

(1) Sensitization, Induction of Arthritis, administration of test substance, and collection of joint fluid

Ovalbumin (SIGMA) was dissolved and prepared with physiological saline (Otsuka Pharmaceutical Factory, Inc.) at 1% (w/v), and a water-in-oil emulsion was prepared with Freund's complete adjuvant (CAPPEL) at 1:1 (v:v). The emulsion was intradermally administered at a total dose of 1 mL to dozens of sites of the back of a Japanese white rabbit (male, 16 weeks of age, Oriental Yeast Industry Co., Ltd.) under general anesthesia (midazolam, xylazine, and butorphanol at 0.67 mg/kg, 5.3 mg/kg, and 0.67 mg/kg, i.v., respectively) to sensitize the rabbit. On day 12 after sensitization, the rabbit was additionally sensitized by administering the emulsion in the same manner. Immediately before administration of the test substance (induction of arthritis) after about 2 to 3 months from the second sensitization, the knee joint cavity of a hind limb of the rabbit under general anesthesia was washed three times with 1 mL of physiological saline, and the joint fluid was collected. Thereafter, the test substance (containing the arthritis-inducing substance) was administered into the knee joint cavity of the hind limb of the rabbit at a dose of 0.5 mL/joint. In a normal group, the Normal solution (not containing the arthritis-inducing substance) was administered, so that arthritis was not induced. At 48 hours after the administration, the knee joint cavity of the hind limb of the rabbit under general anesthesia was washed three times with 1 mL of physiological saline, and the joint fluid was collected. The collected joint fluid was centrifuged, and the supernatant was collected and cryopreserved.

(2) Pronase Treatment

The collected joint fluid was heated at 100° C. for 10 minutes. Thereafter, 30 μL of 200 μg/mL pronase (Merck Co., Ltd.) and 270 μL of the joint fluid were mixed to allow the digestion to proceed overnight at 37° C., whereby a pronase treatment was performed. The joint fluid after the pronase treatment was heated at 100° C. for 10 minutes, followed by centrifugation, and the supernatant was collected and cryopreserved.

(3) Fractionation of Supernatant and Measurement of Radioactivity

Since glucosamine is a constituent monosaccharide of hyaluronic acid, in hyaluronic acid newly synthesized in the living body after administration of the test substance, [³H] glucosamine is incorporated. Therefore, hyaluronic acid in the collected supernatant was separated according to molecular weight using HPLC, and fractions were collected every 0.5 minutes using a fraction collector. To each of the collected fractions, 0.5 mL of a scintillation fluid was added and mixed. Thereafter, the radioactivity (dpm, disintegrations per minute) of each fraction was measured using a scintillation counter, and the radioactivity (the amount of [³H] glucosamine incorporated into newly synthesized hyaluronic acid) was evaluated. The HPLC conditions are as follows.

Mobile phase: a solution of 5 mmol/L phosphate buffer solution (pH 6.0) with 0.82% (w/v) NaCl:acetonitrile=2:1 (v:v)

Flow rate: 0.5 mL/min

Column: OH pak SB-807 HQ column (Shodex (registered trademark)), column temperature: 35° C.

Injection volume: 100 μL

Scintillation counter conditions

³H, dpm, 3 min

Scintillation fluid: Ultima-Flo™ M, cocktail for flow scintillation analyzer

The hyaluronic acid standard solution was separated by HPLC, and UV absorption at a wavelength of 210 nm was measured. Fraction No. in which the peak top of the hyaluronic acid standard solution of each molecular weight is collected was calculated. As standard hyaluronic acid samples, Streptococcal Hyaluronic Acid Polymer (average molecular weight: 804,000, Iduron Ltd.) and Select-HATM 2,500 k (average molecular weight: 2,384,000) were used. Further, the fraction No. was calculated in the same manner also for a join fluid collected from a knee joint of a normal rabbit.

### (Results)

The results are shown in FIG. 7.

(1) control (n=3)

(2) HA (n=3)

(3) compound 1 (n=3)

(4) normal (without inducing arthritis) (n=1)

In FIG. 7, the results are shown as average values (n=3, only in the normal group, n=1). In the case of hyaluronic acid synthesized in the knee joint of the normal rabbit, a peak was observed in a molecular weight range higher than 2,300,000 (normal joint fluid). In the case of hyaluronic acid synthesized in the knee joint of the rabbit after administering the compound 1, a peak was observed in the vicinity of 2,300,000. The amount of high molecular weight hyaluronic acid produced in the compound 1 administration group was prominent as compared with the sodium hyaluronate administration group. Further, the production amount of hyaluronic acid was more prominent in the compound 1 administration group even as compared with the normal group.

Although the present invention has been described in connection with specific Examples and various embodiments, many modifications and applications of the embodiments described herein may be made without departing from the spirit and scope of the invention as will be readily understood by a person skilled in the art.

The present application claims priority based on Japanese Patent Application No. 2018-59772 filed with the Japan Patent Office on Mar. 27, 2018, the contents of which are incorporated herein by reference in their entirety.

1.-6. (canceled)

7. A method for promoting hyaluronic acid synthesis, comprising bringing a polysaccharide derivative represented by the following formula 1 or a salt thereof into contact with hyaluronic acid-producing cells:
[Chem. 5]
XA)ₙ formula 1
wherein X is a residue derived from a polysaccharide having at least one of a carboxy group and a hydroxy group; A is a substituent; n is an introduction ratio of the substituent A and is not less than 1 mol % and not more than 80 mol %; between X and A is a bond of the carboxy group or the hydroxy group and the substituent A, and the bond is selected from the group consisting of an ester, a thioester, and an amide; and the substituent A is represented by the following formula 2:
[Chem. 6]
*—Y—Z formula 2
wherein Y is a spacer residue or an ester bond; Z is a diclofenac residue; when Y is a spacer residue, the bond between Y and Z is selected from the group consisting of an ester, a thioester, and an amide; and * is a binding site to X.

8. The method for promoting hyaluronic acid synthesis according to claim 7, wherein the polysaccharide is selected from the group consisting of hyaluronic acid, chondroitin, chondroitin sulfate, heparin, heparan sulfate, and carboxy C₁₋₄ alkyldextran.

9. The method for promoting hyaluronic acid synthesis according to claim 7, wherein the spacer residue is selected from the group consisting of a C₁₋₆ alkylene group, an amino acid residue, and a polypeptide chain.

10. The method for promoting hyaluronic acid synthesis according to claim 7, wherein the promotion of hyaluronic acid synthesis is an increase in the molecular weight of hyaluronic acid to be synthesized in the hyaluronic acid-producing cells.

11. The method for promoting hyaluronic acid synthesis according to claim 7, wherein the average molecular weight of the polysaccharide is not less than 10,000 and not more than 5,000,000.

12. The method for promoting hyaluronic acid synthesis according to claim 7, wherein the hyaluronic acid-producing cells are selected from the group consisting of synovial cells, chondrocytes, fibroblasts, keratinocytes, smooth muscle cells, oral mucosal cells, vascular endothelial cells, and mammary epithelial cells.

13. A method for evaluating the responsiveness of hyaluronic acid-producing cells to a polysaccharide derivative represented by the following formula 1 or a salt thereof, comprising:
(1) culturing the hyaluronic acid-producing cells in a culture medium containing the polysaccharide derivative or a salt thereof; and
(2) measuring the molecular weight and/or the content of hyaluronic acid in the culture medium:
[Chem. 7]
XA)ₙ formula 1
wherein X is a residue derived from a polysaccharide having at least one of a carboxy group and a hydroxy group; A is a substituent; n is an introduction ratio of the substituent A and is not less than 1 mol % and not more than 80 mol %; between X and A is a bond of the carboxy group or the hydroxy group and the substituent A, and the bond is selected from the group consisting of an ester, a thioester, and an amide; and the substituent A is represented by the following formula 2:
[Chem. 8]
*—Y—Z formula 2
wherein Y is a spacer residue or an ester bond; Z is a diclofenac residue; when Y is a spacer residue, the bond between Y and Z is selected from the group consisting of an ester, a thioester, and an amide; and * is a binding site to X.

14. The method according to claim 13, further comprising: (3) detecting the presence of responsiveness of the hyaluronic acid-producing cells to the polysaccharide derivative or a salt thereof using an increase in the molecular weight and/or the content of hyaluronic acid as an index.

15. A method for producing hyaluronic acid, comprising:
(1′) culturing hyaluronic acid-producing cells in a culture medium containing a polysaccharide derivative represented by the following formula 1 or a salt thereof; and
(2′) collecting hyaluronic acid from the culture medium:
[Chem. 9]
XA)ₙ formula 1
wherein X is a residue derived from a polysaccharide having at least one of a carboxy group and a hydroxy group; A is a substituent; n is an introduction ratio of the substituent A and is not less than 1 mol % and not more than 80 mol %; between X and A is a bond of the carboxy group or the hydroxy group and the substituent A, and the bond is selected from the group consisting of an ester, a thioester, and an amide; and the substituent A is represented by the following formula 2:
[Chem. 10]
*—Y—Z formula 2
wherein Y is a spacer residue or an ester bond; Z is a diclofenac residue; when Y is a spacer residue, the bond between Y and Z is selected from the group consisting of an ester, a thioester, and an amide; and * is a binding site to X.

16.-18. (canceled)

19. The method according to claim 13, wherein the polysaccharide is selected from the group consisting of hyaluronic acid, chondroitin, chondroitin sulfate, heparin, heparan sulfate, and carboxy C₁₋₄ alkyldextran.

20. The method according to claim 13, wherein the spacer residue is selected from the group consisting of a C₁₋₆ alkylene group, an amino acid residue, and a polypeptide chain.

21. The method according to claim 13, wherein the average molecular weight of the polysaccharide is not less than 10,000 and not more than 5,000,000.

22. The method according to claim 13, wherein the hyaluronic acid-producing cells are selected from the group consisting of synovial cells, chondrocytes, fibroblasts, keratinocytes, smooth muscle cells, oral mucosal cells, vascular endothelial cells, and mammary epithelial cells.
